(19) United States
(12) Patent Application Publication
(10) Pub. No.: US 2026/0301180 A1
(43) Pub. Date: Oct. 1, 2026

(54) Method for Providing Evaluation Information from at least one Magnetic Resonance Image

(71) Applicant: Siemens Healthineers AG, Forchheim (DE)

(72) Inventors: Silvia Bettina Arroyo Camejo, Fuerth (DE); Vanya Saksena, Nürnberg (DE); Rainer Schneider, Höchstadt (DE)

(73) Assignee: Siemens Healthineers AG, Forchheim (DE)

(21) Appl. No.: 19/575,233

(22) Filed: Mar. 23, 2026

(30) Foreign Application Priority Data
Mar. 25, 2025 (DE) 10 2025 111 404.8

(51) Int. Cl. G06T 7/00; A61B 5/00; A61B 5/055; G01R 33/56; G06V 10/764; G06V 10/776

(52) U.S. Cl. CPC G06T 7/0014; A61B 5/055; A61B 5/7264; G01R 33/5608; G06V 10/764; G06V 10/776; G06T 2207/10088; G06T 2207/20081; G06T 2207/30016; G06T 2207/30168; G06V 2201/031

(57) ABSTRACT

The disclosure relates to a computer-implemented method for providing evaluation information from at least one magnetic resonance image of a patient including providing the at least one magnetic resonance image of the patient and applying a trained machine-learning model to the at least one magnetic resonance image. The evaluation information comprises a difference map between the at least one magnetic resonance image, and a reference image is ascertained by means of the trained machine-learning model. The difference map depicts a pathological abnormality and/or an abnormality with regard to image quality. The evaluation information is provided and output to a user.

### CROSS-REFERENCE TO RELATED APPLICATIONS

This application claims priority to Germany application no. DE 10 2025 111 404.8, filed on Mar. 25, 2025, the contents of which are incorporated herein by reference in its entirety.

### TECHNICAL FIELD

The present disclosure relates to a computer-implemented method for providing evaluation information from at least one magnetic resonance image. Furthermore, the present disclosure relates to a computer-implemented method for providing a trained machine-learning model. The disclosure is further based on an evaluation module and a computer program product.

### BACKGROUND

Evaluation and/or interpretation of magnetic resonance images is usually very time-consuming and requires extensive experience on the part of the interpreting medical operator, in particular a physician. When interpreting and/or evaluating the magnetic resonance images, medical operators must be able to distinguish pathological abnormalities from possible artifacts in the magnetic resonance images. It may also be difficult for a physician interpreting magnetic resonance images to identify pathological abnormalities if, for example, the magnetic resonance images exhibit a high noise component. It is therefore important to check image quality as early as when capturing magnetic resonance images so that, if the image quality of the captured magnetic resonance images is insufficient for interpretation, new magnetic resonance image data with better image quality can be captured.

### SUMMARY

The present disclosure is based on the object of supporting medical operators in capturing and interpreting magnetic resonance images and providing information for evaluating the captured magnetic resonance images. The object is achieved by the features of the various embodiments as described herein, including the claims.

The disclosure is based on a computer-implemented method for providing evaluation information from at least one magnetic resonance image of a patient, comprising the following steps:
provision of the at least one magnetic resonance image of the patient,
applying a trained machine-learning model to the at least one magnetic resonance image, wherein evaluation information comprising a difference map depicting a difference between the at least one magnetic resonance image and a reference image is ascertained by means of the trained machine-learning model, wherein the difference map depicts a pathological abnormality and/or an abnormality with regard to image quality,
providing the evaluation information, and
outputting the evaluation information to a user.

The method for providing evaluation information from at least one magnetic resonance image is embodied to assist a user, such as a medical operator, for example, a physician, in capturing and/or interpreting magnetic resonance image data from a patient and provide the medical operator with information for evaluating the captured magnetic resonance images. In an embodiment, the evaluation information is embodied to provide the user with information about the magnetic resonance images, wherein the user can use the information to take further steps to process and/or evaluate the magnetic resonance images. For example, the evaluation information can comprise information relating to the image quality of the magnetic resonance data, so that the user can use the evaluation information to decide whether or not the image quality of the magnetic resonance images is sufficient for interpretation and/or diagnosis. Alternatively or additionally, the evaluation information can also comprise information relating to a pathological abnormality identified in the magnetic resonance data.

The method can be performed as early as during data capture, e.g. when capturing magnetic resonance data during a magnetic resonance examination on a patient. This allows a medical operator to decide during the magnetic resonance examination, based on the evaluation information, whether an image quality of the captured image data is sufficient for diagnosis and/or interpretation. Alternatively or additionally, the user can also be shown whether there is a pathological abnormality in the captured image data.

In an embodiment, the method is performed for all magnetic resonance images captured during a measuring program and/or the execution of a measurement step with a magnetic resonance sequence, so that evaluation information is available for all captured magnetic resonance images.

The at least one magnetic resonance image may e.g. be provided by means of a provision module. The provision module may e.g. comprise a data interface by means of which magnetic resonance images are provided for evaluation. Herein, the provision of the at least one magnetic resonance image can take place simultaneously with a magnetic resonance examination of a patient, and thus with the capture of magnetic resonance images, so that the magnetic resonance images provided comprise currently captured magnetic resonance images. Alternatively or additionally, the provision of the at least one magnetic resonance image can also comprise retrieving stored magnetic resonance images held in a memory, for example a database and/or a cloud.

If the method is performed simultaneously with the magnetic resonance examination, then the magnetic resonance examination on the patient is defined before the provision of the at least one magnetic resonance image. Here, e.g., magnetic resonance sequences selected for individual measurement steps are executed sequentially during the magnetic resonance examination, preferably in a defined order. The individual measurement steps, e.g. the individual magnetic resonance sequences, may be tailored to a clinical and/or diagnostic issue to be clarified by means of the magnetic resonance examination on the patient.

In addition, an evaluation module for evaluating the captured magnetic resonance image data can also be activated for the individual measurement steps, e.g. the individual magnetic resonance sequences, provided that an evaluation module is available for these measurement steps, e.g. these magnetic resonance sequences. The evaluation module may be used to ascertain and provide the evaluation information for magnetic resonance image data. Herein, the evaluation module can be actively selected by a user, for example a medical operator and/or physician who determines and/or supervises the magnetic resonance examination. In addition, the evaluation module can also be selected automatically for the individual measurement steps, e.g. the individual magnetic resonance sequences. In addition, it can also be the case that the evaluation module can only be selected when the respective measurement steps, e.g. the respective magnetic resonance sequences, have been executed. For example, it can be the case that, during the capture of the magnetic resonance image data, the medical operator identifies that there is an image quality problem with the data and therefore receives support from the evaluation information in the evaluation of the magnetic resonance image data.

In general, trained machine-learning models (ML models) make predictions and/or issue statements without any justification that is understandable to humans. End users usually find it difficult or impossible to understand what the trained ML model “thought” when ascertaining the result and to efficiently verify whether the result is correct. Particularly in risky applications, such as, for example, the medical sector, it is very important for results to be explainable. The difference map provides a medical operator, for example, a physician or radiologist, with a visual depiction that goes beyond a purely numerical result, for example, a quality value or a quality class, from the ML model. The difference map enables the medical operator to quickly understand and verify the result of a trained ML model. For example, the medical operator can easily query and confirm or reject an image quality evaluation obtained from the trained ML model.

The evaluation information may e.g. be ascertained by means of the evaluation module comprised by the trained machine-learning model (ML model). The evaluation module may e.g. have an input interface that communicates with the provision module, e.g. the data interface of the provision module, and receives the at least one magnetic resonance image from the data interface of the provision module.

Herein, the trained ML model can comprise a conventionally trained ML model. In general, a trained ML model mimics cognitive functions. For instance, training based on training data enables the trained ML model to adapt to new circumstances and identify and extrapolate patterns. Another term for a trained ML model is trained function.

In general, parameters of an ML model can be adapted by means of training. Herein, e.g. supervised training, semi-supervised training, unsupervised training, reinforcement training (reinforcement learning), etc. can be used. In addition, representation learning can be used, wherein representation learning comprises a subfield of machine learning that focuses on the development of algorithms that learn how to best represent data. In an embodiment, the parameters of the ML models can be adapted iteratively by a plurality of training steps. For example, a specific cost function can be minimized during training. In an embodiment, a backpropagation algorithm can be used during the training of a neural network.

In an embodiment, the ML model comprises a deep-learning algorithm to ascertain the evaluation information for the at least one magnetic resonance image. Herein, the deep-learning algorithm uses artificial neural networks (ANNs). Herein, the deep-learning algorithm, e.g. the artificial neural network, comprises an input layer, an output layer and preferably a plurality of hidden layers. A deep-learning algorithm may enable processing and analysis of complex data patterns. For this purpose, deep hierarchical neural networks are used that can extract abstract features from the data.

In an embodiment, the trained ML model is embodied to make image predictions based on image data. For example, such a model can be a GAN model or a CycleGAN model. Advantageously, such a model can be a U-Net model or a Vision Transformer model.

A GAN (generative adversarial network) model describes a framework for training networks in the context of generative learning or unsupervised learning. Here, two networks, a generator and a discriminator, are trained against one other, wherein the generator attempts to generate data that is very similar to an original dataset and the discriminator attempts to distinguish between genuine and spurious data. After training, the generator can be used to generate data that is very similar to the original data.

A U-Net model is a convolutional neural network (CNN) developed for image segmentation. Herein, a neural network is supplemented by successive layers, wherein pooling operations are replaced by upsampling operators. Therefore, these layers increase the resolution of the output. A convolutional layer can then learn to assemble a precise output based on this information.

A transformer model or transformer network is a neural network architecture that generally comprises an encoder or a decoder or both an encoder and a decoder. In some cases, the encoder and/or encoder consist of a plurality of corresponding encoding layers or decoding layers. There may be an attention mechanism within each encoding and decoding layer. The attention mechanism, sometimes referred to as self-attention, relates data elements (pixels) with a row of data elements to other data elements within this row. The self-attention mechanism, for example, enables the model to examine a group of voxels within a medical image and determine the relative importance of other voxel groups within the medical image relative to the voxel group to be examined.

In an embodiment, the encoder of a transformer model or a transformer network can be configured to transform the input (medical image) into a numerical representation. The numerical representation can comprise a vector for each input token (voxel). The encoder can be configured to implement an attention mechanism so that each vector of a token is affected by the other tokens in the input.

In an embodiment, the decoder of a transformer model or transformer network can be configured to transform input into a sequence of output tokens. In an embodiment, the decoder can be configured to implement a masked self-attention mechanism so that each vector of a token is only affected by the other tokens to one side of a sequence. Furthermore, the decoder can be autoregressive, i.e., intermediate results are fed back.

In the context of this disclosure, it has been found that the best results for ascertaining the evaluation information can preferably be achieved by means of a pretrained Vision Transformer model in which the pretrained model parameters were largely frozen and only the last layers were subject to transfer learning on the specific training dataset. Vision transformer models are widely used in image identification tasks, such as, for example, image segmentation, object identification, classification, etc.

In Vision Transformer models, images are depicted as sequences and class labels are predicted for the image. This enables models to learn the image structure independently of one another. Input images of the Vision Transformer models are processed as a sequence of patches. Each of these patches is flattened to a vector by concatenating the channels of all pixels in a patch and then projecting it linearly to the desired input dimension. Herein, image patches are the sequence tokens. Vision transformer models also comprise encoders comprising a plurality of blocks, wherein each block comprises a layer norm, a multi-head attention network (MSP) and a multi-layer perceptron (MLP).

The layer norm of a Vision Transformer model keeps the training process on track and enables the model to adapt to variations among the training images. MSP comprises a network responsible for generating attention maps from the given embedded tokens. These attention maps help the network to focus on the most critical regions in the image, for example, on objects. MLP is a two-layer classification network. MLP is used as the output of the Vision Transformer model.

In an embodiment, the input data of the trained ML model comprises a magnetic resonance image to be evaluated and the output data of the trained ML model comprises the evaluation information. The evaluation information comprises at least the difference map. In addition, the evaluation information ascertained may not be limited to the difference map and can comprise further additional information, such as, for example, a deviation value and/or a measure for image classification. Herein, the deviation value can indicate a value of an average deviation of the magnetic resonance image from an ideal image.

The difference map visually depicts a pathological abnormality and/or abnormality with regard to image quality in the magnetic resonance image for a user, e.g. a medical operator supervising the magnetic resonance examination, for example a physician and/or radiologist. Herein, the ML model may be trained for a defined abnormality, e.g. a defined pathological abnormality and/or an abnormality with regard to a defined image quality criterion. Herein, the depiction in the difference map can be limited to a pathological abnormality and/or abnormality with regard to image quality in the magnetic resonance image, e.g. to the defined pathological abnormality and/or the abnormality with regard to a defined image quality criterion. In other words, herein, only the pathological abnormality and/or abnormality with regard to image quality in the magnetic resonance image can be depicted in the difference map. This limitation of the depiction in the difference map to the pathological abnormality and/or abnormality with regard to image quality in the magnetic resonance image can also be regarded as a marking of the pathological abnormality and/or abnormality with regard to image quality.

This provides the medical operator with an aid that enables an abnormality to be captured quickly and easily when evaluating the evaluation information. Thus, the medical operator can also decide particularly quickly whether the abnormality is located in a region relevant for the interpretation and/or evaluation of the magnetic resonance image or whether, for example, the image quality is sufficient for interpretation and/or evaluation despite the presence of an abnormality.

The reference image may e.g. comprise an “ideal” image comprising no abnormalities or only comprising minor abnormalities, such as artifacts or signal noise or pathological abnormalities. The reference image e.g. may comprise the same image section as the at least one magnetic resonance image. Herein, the reference image can comprise a magnetic resonance image that has been measured and/or captured during the execution of a measurement step. Alternatively, the reference image can also be generated by simulation. Herein, the difference map comprises differences and/or deviations between the reference image and the at least one magnetic resonance image. Herein, the differences and/or deviations between the reference image and the at least one magnetic resonance image are preferably ascertained pixel-by-pixel. Herein, the reference image is only required for training the ML model. The trained ML model can then ascertain the difference map solely from the magnetic resonance image that is obtained and/or provided.

If, for example, the ML model is trained to identify and/or capture motion artifacts in the magnetic resonance images, the reference image or plurality of reference images should not exhibit any motion artifacts. However, the reference image or the plurality of reference images can contain other characteristics of a natural data distribution, for example, a wide variety of pathologies and/or other features that affect image quality, such as, for example, a high noise component in the captured data. If, on the other hand, the ML model is trained to identify and/or capture a specific and/or defined pathological abnormality in the magnetic resonance images, the reference image or the plurality of reference images should not exhibit this pathological abnormality. Instead, the reference image or the plurality of reference images could also contain other characteristics of a natural data distribution, such as, for example, motion artifacts or a high noise component or other pathological abnormalities that do not comprise the defined pathological abnormality.

The evaluation information may e.g. be provided by means of the evaluation module, which has an output interface for this purpose. In an embodiment, the evaluation information is provided for output of the evaluation information. The evaluation information may e.g. be output to the user by means of an output unit, e.g. a visual output unit, such as for instance a monitor and/or a display.

The disclosure has the advantage that a medical operator, for example, a physician, can be advantageously assisted in the evaluation and/or interpretation of magnetic resonance image data. In an embodiment, evaluation information can be provided as early as during the capture of magnetic resonance data for access by the medical operator or for visual display to the medical operator. This allows the medical operator to be alerted directly during the capture of the magnetic resonance data to abnormalities and/or differences in the magnetic resonance image data that are unsuitable for interpretation and/or diagnosis. This enables the medical operator to decide whether individual measurement steps should be repeated or whether alternative measurement steps should be executed to capture magnetic resonance data. In an embodiment, the difference map enables the medical operator to quickly gain an overview of the at least one magnetic resonance image and thus of the current measurement step.

A further advantage of the disclosure is that a trained ML model provides a tool that can be used to advantageously increase the speed and/or efficiency of the provision of evaluation information from at least one magnetic resonance image. In an embodiment, the evaluation information can be provided by the trained ML model immediately after the capture of the at least one magnetic resonance image and thus during the performance of the measurement step. In an embodiment, the difference map enables a medical operator to easily understand or verify a result of the trained ML model. If only a quality value or quality class were output, it would be difficult for the medical operator to verify the result on the basis of the magnetic resonance image, since trained ML models or trained deep-learning models naturally do not provide explanations as to how and/or why they arrived at a specific result. Therefore, predictions or results from trained ML models or trained deep-learning models are not easily comprehensible to an operator and/or user. The difference map has the great advantage that it makes the evaluation result of the trained ML model comprehensible and displays it visibly to the user.

An advantageous development of the method according to the disclosure for providing evaluation information from at least one magnetic resonance image can provide that the trained machine-learning model is trained to ascertain and provide a difference map for at least one of the following applications:
at least one defined pathological abnormality in the at least one magnetic resonance image and/or
an abnormality with regard to at least one defined image quality criterion in the at least one magnetic resonance image.

The at least one defined pathological abnormality may e.g. comprise a specific pathological abnormality, for example, the presence of a tumor and/or bleeding and/or injuries and/or diseases, etc., for which the ML model was trained. In an embodiment, the ML model was trained for different manifestations of the specific pathological abnormality. The at least one specific pathological abnormality is also measurement-step-dependent and/or measurement-sequence-dependent, so that when a measurement step and/or a measurement sequence with which the at least one magnetic resonance image provided was captured is selected, a trained ML model that can identify specific pathological abnormalities in these magnetic resonance images is also provided. For example, when selecting a measurement step for a head examination, the ML model can be trained to identify deviations with regard to bleeding and/or tumors. In addition, the ML model can also be trained for two or more specific pathological abnormalities in the at least one magnetic resonance image.

Herein, the at least one defined image quality criterion comprises a specific image quality criterion, such as, for example, background noise or motion artifacts, etc., in the at least one magnetic resonance image. In an embodiment, the ML model was trained for different manifestations of the specific image quality criterion. For example, the ML model can be trained for magnetic resonance image data from different magnetic resonance sequences with respect to different image quality criteria. For example, for magnetic resonance sequences that are particularly sensitive to background noise in the magnetic resonance images, the ML model be trained with regard to background noise in the magnetic resonance images. In addition, for magnetic resonance sequences in which undesirable patient movement is common, the ML model can be trained with regard to motion artifacts in the magnetic resonance images.

It is also possible for a plurality of ML models be trained with respect to different defined pathological abnormalities and/or different defined image quality criteria to be able to provide a comprehensive evaluation of the magnetic resonance image. Herein, each individual ML model can be specifically trained to identify one or a few pathological abnormalities and/or to identify a defined image quality criterion or a plurality of different defined image quality criteria. In this way, the evaluation information provided for the magnetic resonance image can cover multiple critical image aspects, e.g. a plurality of defined pathological abnormalities and/or a plurality of different defined image quality criteria, and hence alert the medical operator to various potential objections or suspected diagnoses. Alternatively, the ML model can be trained with respect to different defined pathological abnormalities and/or different defined image quality criteria.

In this way, specific image quality problems in the magnetic resonance image data can be identified at an early stage or the medical operator can be made aware of these problems at an early stage. In addition, the medical operator can be made aware of specific pathological abnormalities when evaluating and/or interpreting the captured magnetic resonance image data.

Input training data and output training data are provided for training the ML model. The input training data may e.g. comprise training magnetic resonance images. The output training data may e.g. comprise training difference maps. During the training of the ML model, a training difference map, e.g. a corresponding training difference map, which was created and/or ascertained from the training magnetic resonance image, is generated for each training magnetic resonance image, and provided to the ML model.

The provision of training difference maps requires reference images. These reference images basically represent “good images” and may e.g. comprise ideal images from magnetic resonance recordings. Such reference images exhibit no abnormalities or exhibit hardly any abnormalities. These reference images can comprise a measured magnetic resonance image. Alternatively, these reference images can also be generated for this purpose using a simulation.

Herein, the training magnetic resonance images and the reference images comprise the same image section and/or the same settings in the parameter settings for capturing the image data, thereby making it easy to compare the at least one magnetic resonance image and the reference image. Herein, the training magnetic resonance images can be simulated from the reference images, e.g. from the “good images”, by means of artifact simulation and/or pathology simulation. These training magnetic resonance images basically represent “bad images” and may e.g. comprise at least one abnormality. In an embodiment, the abnormalities are contained in the different training magnetic resonance images in different manifestations with regard to a specific image quality criterion, for example from low signal noise to high signal noise. In addition, the abnormalities are also contained in the different training magnetic resonance images in different manifestations with regard to a specific pathological problem, for example, tumors of different sizes.

Each training difference map represents a difference between a reference image, e.g. a “good image”, and a training magnetic resonance image, e.g. a “bad image”. The training difference map may for instance be ascertained using at least one of the following difference metrics and/or difference algorithms:
NRMS (normalized root mean square): pixel-by-pixel determination of the difference between “good image” and “bad image”,
MSE (mean squared error),
SSIM (structural similarity index): consideration of parameters such as, for example, contrast and structural information between the reference image and the magnetic resonance image to determine a deviation and/or a difference.

PSNR (peak signal-to noise-ratio): ratio between the signal strength and noise level in an image, indicating the quality of the image, and
LPIPS (learned perceptual image patch similarity): assessment of the perceptual similarity between two images.

Further pixel-based standard image quality metrics and/or difference algorithms for determining the training difference map are conceivable at any time.

An advantageous development of the method according to the disclosure for providing evaluation information from at least one magnetic resonance image can provide that the abnormality with regard to at least one defined image quality criterion comprises signal noise and/or a motion artifact and/or Gibbs ringing. In this way, specific image quality criteria that make it difficult or impossible to interpret and/or diagnose the magnetic resonance image data can be identified in good time during the magnetic resonance examination on the patient. A further advantage is that, if such abnormalities in the magnetic resonance image data are identified at an early stage, corrective means can be initiated by a medical operator in good time. For example, individual measurement steps of a magnetic resonance examination can be repeated or repeated with different parameter settings or a different magnetic resonance sequence can be selected for the measurement step to obtain magnetic resonance image data with high image quality.

Gibbs ringing, also known as Gibbs artifacts, can appear in magnetic resonance images as multiple fine parallel lines immediately adjacent to high-contrast interfaces. These artifacts result from overshoots at e.g. high-contrast interfaces in the image data. These artifacts are particularly problematic in spinal imaging, in which they may artificially widen or narrow the spinal cord or mimic syringomyelia.

An advantageous development of the method according to the disclosure for providing evaluation information from at least one magnetic resonance image can provide that a deviation value is calculated from the difference map, wherein the deviation value represents a measure of a pathological abnormality and/or image quality and wherein the deviation value is comprised by the evaluation information and can be output to the user. For example, such a deviation value can be calculated from a mean value of all pixel values of the difference map. Herein, the deviation value can comprise a value between 0 and 1. Herein, a value of 0 or close to 0 can indicate a small deviation or a minor abnormality in the difference map, while a value of 1 or close to 1 can indicate a major deviation or a major abnormality in the difference map. In addition, the deviation value can also comprise a value between −1 and 1. This embodiment of the disclosure has the advantage that a medical operator can obtain a particularly quick evaluation of the magnetic resonance image, e.g. with regard to an image quality criterion of the magnetic resonance image, without herein having to interpret the difference map.

An advantageous development of the method according to the disclosure for providing evaluation information from at least one magnetic resonance image can provide that an image classification of the at least one magnetic resonance image is ascertained on the basis of the deviation value, wherein the image classification comprises at least two image classes. The at least two image classes represent the simplest type of image classification and, herein, can comprise the image class “good” and the image class “bad”. Herein, the image class “good” can be assigned the deviation value 0, wherein a range of the deviation value assigned to the image class “good” can be dependent on a magnetic resonance sequence and/or a type of interpretation, etc. The range for the image class “good” can, for example, be between 0 and 0.49 or also between 0 and 0.35. The image class “bad” can be assigned the deviation value 1, wherein a range of the deviation value assigned to the image class “bad” can be dependent on a magnetic resonance sequence and/or a type of interpretation etc. The range for the image class “bad” can, for example, be between 0.5 and 1 or also between 0.35 and 1.

A differentiated image classification can exhibit three image classes, wherein the individual image classes can be divided into “good”, “intermediate” and “bad”. Herein, the image class “good” can be assigned a deviation value of 0, the image class “intermediate” can be assigned a deviation value of 0.5 and the image class “bad” can be assigned a deviation value of 1. Here, once again, ranges for the deviation value can be assigned to the individual image classes, which can likewise be dependent on a magnetic resonance sequence and/or a type of interpretation, etc. In addition, further image classes can be available for a differentiated image classification. For example, an image class can be arranged between “good” and “intermediate” and e.g. assigned a deviation value of 0.3. In addition, a further image class can be arranged between “intermediate” and “bad” and may e.g. be assigned a deviation value of 0.7. Here, once again, ranges for the deviation value can be assigned to the individual image classes that can likewise depend on a magnetic resonance sequence and/or a type of interpretation etc.

In addition, the individual image classes of the image classification can also be depicted using different colors. For example, the image class “bad” can be depicted with the color red, the image class “good” with the color green and the image class “intermediate” with the color yellow. In addition, other color selections that appear useful to the person skilled in the art can be used or made available for the image classification.

This embodiment of the disclosure enables even untrained operators, e.g. those responsible for capturing magnetic resonance data during a magnetic resonance examination, to evaluate the captured magnetic resonance images easily and quickly. In addition, experienced medical operators can assess the magnetic resonance images based on a differentiated image classification and/or the deviation value. For example, the assessment can determine whether, although the magnetic resonance images exhibit abnormalities with respect to image quality, they still enable interpretation and/or evaluation.

An advantageous development of the method according to the disclosure for providing evaluation information from at least one magnetic resonance image can provide that the depiction in the difference map and/or the ascertaining of the deviation value and/or the image classification is limited to a relevant region of the difference map. The relevant region preferably comprises a mapped organ and/or an organ to be examined, part of a mapped organ and/or organ to be examined, a group of mapped organs, a defined mapped body region and/or body region to be examined or even all mapped anatomical structures, but without background. Herein, the relevant region can be defined in advance by a user, for example, a physician, for example it can be limited to a region relevant for the examination. It can also be the case that the relevant region is selected depending on a measurement step, e.g. a magnetic resonance sequence. Herein, the selection can also be made at least partially automatically by the trained ML model.

This embodiment of the disclosure has the advantage that only abnormalities and/or deviations within the relevant region are included and considered when ascertaining the deviation value and/or the image classification. This also increases the informative value of the deviation value and/or the image classification, thereby providing the medical operator with reliable assistance for evaluating the at least one magnetic resonance image. In addition, a depiction of the difference map that is limited to the relevant region enables relevant deviations of the at least one magnetic resonance image to be captured easily. This enables even inexperienced medical operators to quickly evaluate the at least one magnetic resonance image.

Furthermore, the disclosure is based on a computer-implemented method for providing a trained machine-learning model, comprising:
receiving input training data,
receiving output training data, wherein the output training data is related to the input training data,
training the machine-learning model based on the input training data and the output training data, wherein the trained machine-learning model is embodied to ascertain evaluation information from at least one magnetic resonance image with regard to a deviation from a reference image, and
providing the machine-learning model for use in a method for providing evaluation information from at least one magnetic resonance image of a patient.

As explained above, it is hence possible to use supervised learning that is known per se, in which e.g. a cost function that depends on a distance measure between the output data ascertained by the model on the basis of the input training data and the output training data can be minimized. The parameters of the trained model can then be iteratively adapted in order to minimize the cost function for a respective batch of training data. For example, gradient optimization algorithms or further optimization algorithms can be used for this purpose.

In an embodiment, the trained ML model is embodied to make and provide image predictions based on image data. For example, such a model can be a GAN model or a CycleGAN model. Advantageously, such a model is a U-Net model or a Vision Transformer model.

The provision of a trained ML model can advantageously increase the speed and/or efficiency of the provision of evaluation information from at least one magnetic resonance image. In an embodiment, the evaluation information can be provided by means of the trained ML model immediately after the capture of the at least one magnetic resonance image and thus during the performance of the measurement step. In addition, this enables the ML model to be efficiently adapted to different problems in different measurement steps and/or different magnetic resonance sequences. For example, for measurement steps and/or magnetic resonance sequences in which motion artifacts frequently occur due to unwanted movements of the patient, the ML model can be trained quickly and efficiently with regard this problem. For measurement steps and/or magnetic resonance sequences in which strong background noise, signal noise or Gibbs ringing frequently occur, the ML model can be trained quickly and efficiently with regard to this problem. In addition, the ML model can be quickly and efficiently adapted with regard to the detection of abnormalities in the captured image data for different measurement steps and/or different magnetic resonance sequences.

The provision of such a trained ML model enables a medical operator to decide quickly whether the captured magnetic resonance data has sufficient image quality for interpretation and/or evaluation. In an embodiment, the medical operator can use the trained ML model to make such a decision while performing a magnetic resonance examination on the patient and thereby accelerate the capture and/or provision of interpretable magnetic resonance data, or even enable it in the first place.

The advantages of the method according to the disclosure for providing a trained machine-learning model can also substantially correspond to the advantages of the method according to the disclosure for providing evaluation information from at least one magnetic resonance image of a patient. The advantages are described in detail above. Features, advantages or alternative embodiments mentioned here can likewise be transferred to the other claimed subject matter and vice versa.

An advantageous development of the method according to the disclosure for providing a trained machine-learning model can provide that the input training data comprises a training magnetic resonance image and the output training data comprises a training difference map. Preferably, the training magnetic resonance image exhibits an abnormality with regard to an image quality problem and/or with regard to a pathological abnormality. In the training phase, the ML model is provided with a corresponding training difference map for each training magnetic resonance image, wherein the corresponding training difference map represents a difference and/or deviation of the training magnetic resonance image from a reference image and/or ideal image. Herein, the ML model is trained to generate and provide a difference map for a magnetic resonance image to enable a medical operator, for example, a physician, to quickly evaluate the captured magnetic resonance data. The learning difference maps provided by the ML model during training of the ML model are correlated with the training difference maps so that a difference between the learning difference maps ascertained during the training and the training difference maps is minimized.

In this manner, an efficient trained ML model can be provided for predicting a difference map for a magnetic resonance image.

An advantageous development of the method according to the disclosure for providing a trained machine-learning model can provide that a reference image is generated to provide the training difference map, wherein the training difference map is generated from a difference between the training magnetic resonance image and the reference image by means of a difference algorithm or a difference metric.

These reference images basically represent “good images” and may e.g. comprise ideal images from magnetic resonance recordings and exhibit no abnormalities or exhibit hardly any abnormalities. Herein, these reference images can be generated by means of a simulation. In addition, the reference images can also comprise image data captured by means of a magnetic resonance apparatus. Herein, the reference image is only needed for training the ML model. Herein, the reference image is only used to create the output training data, for instance the training difference map. The trained ML model can then ascertain the difference map solely from the received and/or provided magnetic resonance image.

If, for example, the ML model is trained to identify and/or capture motion artifacts in the magnetic resonance images, the reference image or plurality of reference images should not exhibit any motion artifacts. However, the reference image or the plurality of reference images can contain other characteristics of a natural data distribution, e.g. also a wide variety of pathologies and/or other features influencing the image quality, such as, for example, a high noise component in the captured data. If, on the other hand, the ML model is trained to identify and/or capture a specific and/or defined pathological abnormality in the magnetic resonance images, the reference image or the plurality of reference images should not exhibit this specific pathological abnormality. Conversely, the reference image or the plurality of reference images can contain other characteristics of a natural data distribution, such as, for example, motion artifacts or a high noise component or also other pathological abnormalities, that do not, however, comprise the defined pathological abnormality.

Furthermore, the training magnetic resonance images are also used to provide the training difference map. These training magnetic resonance images basically represent “bad images” and may e.g. comprise abnormalities. In an embodiment, the abnormalities are contained in the different training magnetic resonance images in different manifestations with regard to a specific image quality criterion, for example, from low background noise or signal noise to high background noise or signal noise or from small motion artifacts to large motion artifacts. In addition, the abnormalities are contained in the different training magnetic resonance images in different manifestations with regard to a specific pathological problem, for example, tumors of different sizes or injuries of varying severity etc. Herein, the training magnetic resonance images can be generated on the basis of the reference images with the corresponding specific abnormality by means of a simulation algorithm, so that comparable and/or corresponding training magnetic resonance images are available for the reference images for the creation of the training difference maps. In an embodiment, the training magnetic resonance image and the reference image comprise the same image section and/or same settings in the sequence parameters of the magnetic resonance sequence and/or the measurement step.

Herein, each training difference map represents a difference between a reference image, e.g. a “good image,” and a training magnetic resonance image corresponding to the reference image, e.g. a “bad image.” Herein, at least one of the following difference algorithms and/or one of the following difference metrics can be used to ascertain the training difference map:
NRMS (normalized root mean square): pixel-by-pixel determination of the difference between “good image” and “bad image”,
MSE (mean squared error),
SSIM (structural similarity index): consideration of parameters such as, for example, contrast and structural information between the reference image and the magnetic resonance image to determine a deviation and/or a difference.

PSNR (peak signal-to noise-ratio): ratio between the signal strength and the noise level in an image, indicating the quality of the image, and
LPIPS (learned perceptual image patch similarity): assessment of the perceptual similarity between two images.

Further pixel-based standard difference algorithms that appear useful to the person skilled in the art for determining the training difference map are conceivable at any time.

An advantageous development of the method according to the disclosure for providing a trained machine-learning model can provide that at least one gradient optimization algorithm is applied to ascertain a learning difference map.

Examples of gradient optimization algorithms are:
Adam (Adaptive Moment Estimation):
Adam is an optimization algorithm used for gradient-based optimization of objective functions, e.g. in deep learning. Adam uses exponentially weighted moving averages of both the first and second moments of the gradients for each parameter.

AdaMax (variant of Adam based on infinity norm):
AdaMax is a first order gradient-based optimization method.

SGD (Stochastic Gradient Descent):
SGD is an iterative method for optimizing an objective function with suitable smoothness properties (e.g., differentiable or subdifferentiable). It can be regarded as a stochastic approximation of gradient descent optimization, since it replaces the actual gradient (calculated from the entire dataset) by an estimate thereof (calculated from a randomly selected subset of the data).
RMSprop (Root Mean Square Propagation):
RMSprop is an optimization algorithm that scales the learning rate for each parameter based on the magnitude of most recent gradients for that parameter.

In addition, for this purpose, further state-of-the-art difference algorithms or difference metrics can be applied to the learning difference map and the training difference map as the main contribution to the training loss. Examples of such difference algorithms or difference metrics include:
NRMS (normalized root mean square): pixel-by-pixel determination of the difference between “good image” and “bad image”,
MSE (mean squared error),
SSIM (structural similarity index),
PSNR (peak signal-to noise-ratio), and
LPIPS (learned perceptual image patch similarity).

Further pixel-based standard difference algorithms for determining the learning difference that map that appear useful to the person skilled in the art are conceivable at any time.

An advantageous development of the method according to the disclosure for providing a trained machine-learning model can provide that the training magnetic resonance image exhibits an abnormality relative to a reference image, wherein the training magnetic resonance image is simulated from the reference image by means of artifact simulation and/or pathology simulation. Artifact simulation and/or pathology simulation can be used to simulate one or more defined abnormalities in the training magnetic resonance images. In an embodiment, such pairs of corresponding images, for instance a reference image or “good image” and a training magnetic resonance image or “bad image”, can be provided for the provision of the training difference map. Advantageously, these pairs of corresponding images have the same image region of an anatomical structure or the same values in the parameter settings of the underlying measurement sequence or the underlying measurement step. For example, in this way a clear assignment of a defined abnormality, e.g. a defined pathological abnormality and/or an abnormality with regard to a defined image quality criterion, to a difference and/or deviation between the training magnetic resonance image and the reference image, e.g. the training difference map, can be achieved in the training magnetic resonance images.

An advantageous development of the method according to the disclosure for providing a trained machine-learning model can provide that the reference image comprises the same image section as the training magnetic resonance image and/or that the reference image is based on the same sequence parameters as the training magnetic resonance image. For example, the reference image and the corresponding training magnetic resonance image have the same contrast settings in the sequence parameters. In addition, further sequence parameters of the reference image and the corresponding training magnetic resonance image can be matched to one another. In this way, training difference maps with a clear abnormality can be provided, wherein the clear abnormality can be assigned to at least one defined image quality problem and/or at least one pathological abnormality.

An advantageous development of the method according to the disclosure for providing a trained machine-learning model can provide that the input training data exhibits a defined image quality problem and/or a defined pathological abnormality. In this way, the ML model can be trained specifically for a defined problem in the magnetic resonance data. In an embodiment, the abnormalities with regard to a defined image quality problem or image quality criterion comprise background noise or signal noise and/or motion artifacts and/or Gibbs ringing, etc. Herein, the defined image quality problem can be manifested in the input training data with varying degrees of severity. In an embodiment, the abnormalities with regard to a defined pathological abnormality comprise, for example, the presence of a tumor and/or bleeding and/or injuries and/or diseases, etc. Herein, the defined pathological abnormality can be manifested in the input training data with varying degrees of severity.

Furthermore, the disclosure is based on an evaluation module comprising the trained machine-learning model for application in the method for providing evaluation information from at least one magnetic resonance image, comprising:
an input interface embodied to receive at least one magnetic resonance image,
a computing unit embodied to apply the trained machine-learning model to the at least one magnetic resonance image, wherein evaluation information comprising a difference map is ascertained by means of the trained machine-learning model,
an output interface embodied to provide the evaluation information.

The input interface of the evaluation module may also be embodied to receive the input training data, e.g. the training magnetic resonance images, and output training data, e.g. the training difference map, during training of the ML model.

The computing unit may for instance comprise a processor that is embodied to apply the trained ML model to the at least one magnetic resonance image. For example, the computing unit may be embodied to execute computer-readable instructions to apply the trained ML model to the at least one magnetic resonance image. The computing unit can be comprised by a magnetic resonance apparatus, for example, a control unit of the magnetic resonance apparatus. In addition, the computing unit can also be embodied separately from the magnetic resonance apparatus and form a separate unit. Furthermore, the computing unit can also be comprised by a cloud.

The advantages of the evaluation module according to the disclosure substantially correspond to the advantages of the method according to the disclosure for providing evaluation information from at least one magnetic resonance image of a patient. The advantages are described in detail above. Features, advantages or alternative embodiments mentioned here can likewise be transferred to the other claimed subject matter and vice versa.

Furthermore, the disclosure is based on a computer program product, which comprises a program and can be loaded directly into a memory of a programmable computing unit, with program means for executing a method for providing evaluation information from at least one magnetic resonance image when the program is executed in the computing unit. The computer program product according to the disclosure can be loaded directly into a memory of a programmable computing unit and has program code means for executing any of the methods according to the disclosure when the computer program product is executed in the computing unit. The computer program product can be a computer program or comprise a computer program. This allows any of the methods according to the disclosure to be carried out quickly, identically repeatedly and robustly. The computer program product is configured such that it can execute the method steps according to the disclosure by means of the computing unit. Herein, the computing unit must in each case fulfill the requisite conditions such as, for example, having an appropriate random-access memory, an appropriate graphics card or an appropriate logic unit so that the respective method steps can be executed efficiently. The computer program product is, for example, stored on a computer-readable medium or held on a network or server from where it can be loaded into the processor of a local computing unit which is directly connected to the magnetic resonance apparatus or can be embodied as part thereof. Furthermore, control information of the computer program product can be stored on an electronically readable data carrier. The control information of the electronically readable data carrier can be embodied to execute any of the methods according to the disclosure when the data carrier is used in a computing unit. Thus, the computer program product can also represent the electronically readable data carrier. Examples of electronically readable data carriers are DVDs, magnetic tapes, hard disks or USB sticks, on which electronically readable control information, in particular software (see above), is stored. When this control information (software) is read from the data carrier and stored in a control unit and/or computing unit, any of the embodiments according to the disclosure of the above-described methods can be performed. Thus, the disclosure can also originate from said computer-readable medium and/or said electronically readable data carrier.

### BRIEF DESCRIPTION OF THE DRAWINGS

Further advantages, features and details of the disclosure will become apparent from the exemplary embodiment described below and from the drawings, in which:

FIG. 1 illustrates an example magnetic resonance apparatus with an example evaluation module in a schematic depiction, in accordance with one or more embodiments of the present disclosure;

FIG. 2 illustrates an example method for providing evaluation information from at least one magnetic resonance image, in accordance with one or more embodiments of the present disclosure;

FIG. 3 illustrates an example trained ML model with input data and output data, in accordance with one or more embodiments of the present disclosure;

FIG. 4 illustrates an example magnetic resonance image of the head, an example reference image, and an example associated difference map, in accordance with one or more embodiments of the present disclosure;

FIG. 5 illustrates an example artifact-free magnetic resonance image of a head of a patient with an associated difference map, in accordance with one or more embodiments of the present disclosure;

FIG. 6 illustrates an example magnetic resonance image of the head of the patient corresponding to FIG. 5 with motion artifacts and an example associated difference map, in accordance with one or more embodiments of the present disclosure;

FIG. 7 illustrates an example method for providing a trained machine-learning model, in accordance with one or more embodiments of the present disclosure;

FIG. 8 illustrates an example reference image and an example corresponding magnetic resonance image with simulated signal noise, in accordance with one or more embodiments of the present disclosure;

FIG. 9 illustrates an example of different difference maps of the reference image and corresponding magnetic resonance image from FIG. 8, in accordance with one or more embodiments of the present disclosure;

FIG. 10 illustrates an example reference image and an example corresponding magnetic resonance image with simulated Gibbs ringing, in accordance with one or more embodiments of the present disclosure;

FIG. 11 illustrates an example of different difference maps of the reference image and corresponding magnetic resonance image from FIG. 10, in accordance with one or more embodiments of the present disclosure; and

FIG. 12 illustrates an example evaluation module with the trained ML model in a schematic depiction, in accordance with one or more embodiments of the present disclosure.

### DETAILED DESCRIPTION

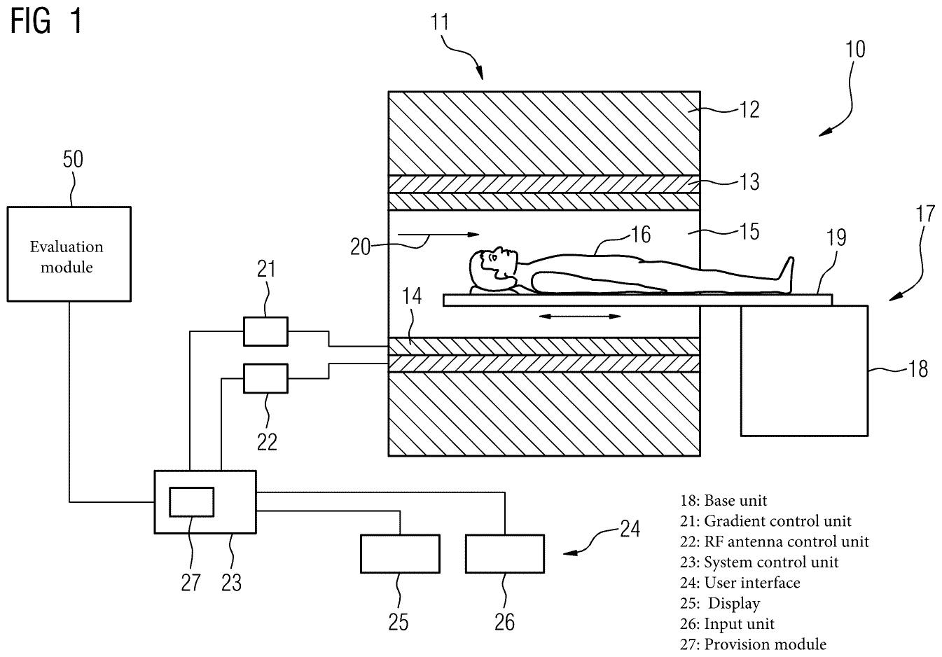

FIG. 1 is a schematic depiction of a magnetic resonance apparatus 10. The magnetic resonance apparatus 10 comprises a magnet unit 11 having a main magnet 12, a gradient coil unit 13 and a radio-frequency antenna unit 14. In addition, the magnetic resonance apparatus 10 has a patient receiving region 15 for receiving a patient 16 for a magnetic resonance examination. In the present exemplary embodiment, the patient receiving region 15 is cylindrical in shape and is surrounded in a circumferential direction by the magnet unit 11 in a cylindrical manner. In principle, however, a different embodiment of the patient receiving region 15 is conceivable at any time.

For positioning the patient 16, e.g. a region of interest of the patient 16, within the patient receiving region 15, the magnetic resonance apparatus 10 has a patient positioning apparatus 17. The patient positioning apparatus 17 has a base unit 18 and a patient table 19 that can be moved relative to the base unit 18. For positioning the patient 16, e.g. the region of interest of the patient 16, the patient table 19 is embodied as movable within the patient receiving region 15. Here, e.g., the patient table 19 is mounted so that it can be moved in the direction of a longitudinal extension of the patient receiving region 15 and/or in the z-direction.

The main magnet 12 of the magnet unit 11 is embodied to generate a strong and constant main magnetic field 20. Herein, the main magnet 12 can, for example, be embodied as a superconducting main magnet 12 or also as a permanent magnet. The gradient coil unit 13 of the magnet unit 11 is embodied to generate magnetic field gradients that are used for spatial encoding during imaging. The gradient coil unit 13 is controlled by means of a gradient control unit 21 of the magnetic resonance apparatus 10. The radio-frequency antenna unit 14 of the magnet unit 11 is embodied to excite polarization that is established in the main magnetic field 20 generated by the main magnet 12. The radio-frequency antenna unit 14 is controlled by a radio-frequency antenna control unit 22 of the magnetic resonance apparatus 10 and emits radio-frequency magnetic resonance sequences into the patient receiving region 15 of the magnetic resonance apparatus 10.

To control the main magnet 12, the gradient control unit 21 and to control the radio-frequency antenna control unit 22, the magnetic resonance apparatus 10 has a system control unit 23. The system control unit 23 centrally controls the magnetic resonance apparatus 10, for example, by performing a predetermined imaging gradient echo sequence. In addition, the system control unit 23 comprises an evaluation unit (not shown in further detail) for evaluating medical image data captured during the magnetic resonance examination.

Furthermore, the magnetic resonance apparatus 10 comprises a user interface 24 connected to the system control unit 23. Control information such as, for example, imaging parameters, and reconstructed magnetic resonance images can be displayed on a display unit 25, for example, on at least one monitor of the user interface 24 for a medical operator. Furthermore, the user interface 24 has an input unit 26, by means of which information and/or parameters can be entered by a medical operator during a measurement procedure.

The magnetic resonance apparatus 10 depicted can obviously comprise further components that are usually comprised by magnetic resonance apparatuses 10. In addition, the general mode of operation of a magnetic resonance apparatus 10 is generally known to the person skilled in the art and so there will no detailed description of the further components.

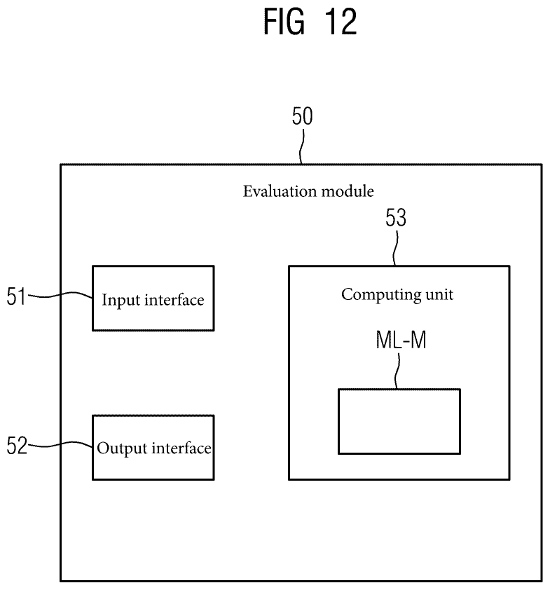

FIG. 1 also depicts an evaluation module 50 (also referred to herein as an evaluation system or a computing system). In the present exemplary embodiment, the evaluation module 50 is embodied as a separate unit from the magnetic resonance apparatus 10, but this is by way of example and not limitation. The evaluation module 50 has an input interface 51, an output interface 52, and a computing unit, as depicted in more detail in FIG. 12.

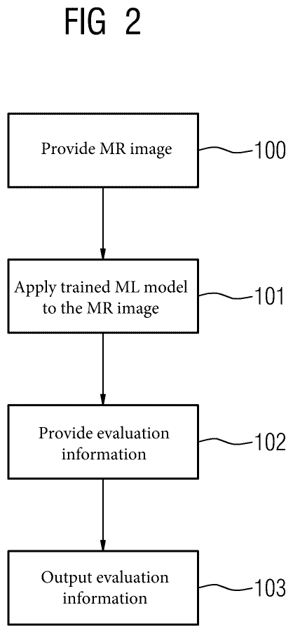

FIG. 2 depicts a method for providing evaluation information from at least one magnetic resonance image MB of a patient. The method can be performed during data capture, e.g. during a magnetic resonance examination, on a patient.

In a first method step 100, the at least one magnetic resonance image MB is provided. The at least one magnetic resonance image MB is provided by means of a provision module 27. Herein, the provision module 27 is comprised by the magnetic resonance apparatus 10, e.g. the system control unit 23 of the magnetic resonance apparatus 10. Herein, provision can take place simultaneously with a magnetic resonance examination of a patient, and thus with the capture of magnetic resonance image data, so that the provided magnetic resonance images MB comprise currently captured magnetic resonance images MB. Alternatively or additionally, the provision of the at least one magnetic resonance image MB can also comprise retrieving stored magnetic resonance image data held in a memory, for example, a database and/or a cloud.

If the method is performed simultaneously with the magnetic resonance examination, the magnetic resonance examination on the patient 16 is scheduled before the provision of the magnetic resonance images MB. The magnetic resonance examination is intended to contribute to the clarification of a diagnostic and/or clinical question. In an embodiment, individual measurement steps and/or magnetic resonance sequences of the magnetic resonance examination that relate to the diagnostic and/or clinical question are selected. In addition, an evaluation module 50 for evaluating the magnetic resonance image data is also selected and/or activated for the individual measurement steps, e.g. for at least one measurement step. In addition, it can also be the case that the evaluation module 50 for evaluating the magnetic resonance image data is also only selected and/or activated when the respective measurement steps, e.g. the respective magnetic resonance sequences, are executed. Herein, the selection and/or activation of the evaluation module 50 for evaluating the magnetic resonance image data can be actively performed by a user, for example, a medical operator. In addition, automatic selection of the evaluation module 50 for evaluating the magnetic resonance image data for the individual measurement steps, e.g. the individual magnetic resonance sequences, is also possible. Herein, the evaluation module 50 is adapted to the individual measurement steps, e.g. magnetic resonance sequences.

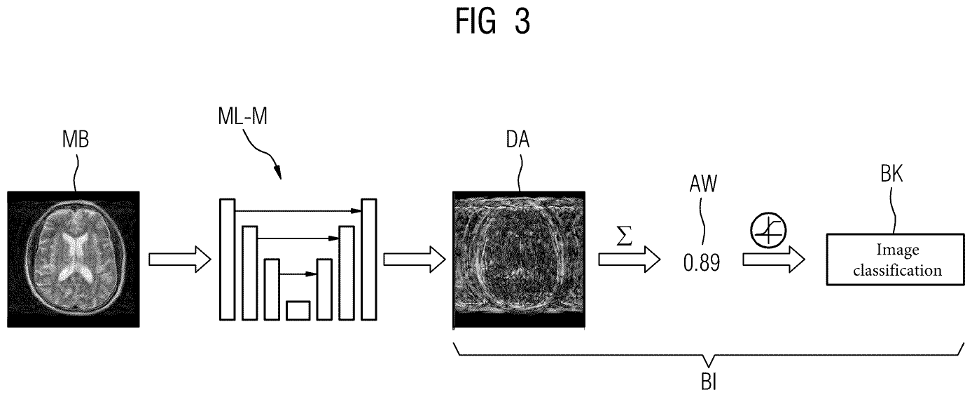

In a further second method step 101, a trained machine-learning model (ML model) ML-M is applied to the at least one magnetic resonance image MB, wherein evaluation information BI comprising a difference map DA between the at least one magnetic resonance image MB and a reference image RB is ascertained by means of the trained ML model ML-M. FIG. 3 depicts the ascertaining of the evaluation information BI with the trained ML model ML-M. Herein, input data of the trained ML model ML-M comprises the at least one magnetic resonance image MB. Output data of the trained ML model ML-M comprises the evaluation information BI comprising the difference map DA. The evaluation information BI may e.g. be ascertained by means of the evaluation module 50, which comprises the trained ML model ML-M. The evaluation module 50 communicates with the provision module 27 via the input interface 51 and receives the at least one magnetic resonance image MB from the provision module 27.

The trained ML model ML-M may e.g. be embodied to make image predictions based on image data. For example, such an ML model ML-M can be a GAN model or a CycleGAN model. Advantageously, such an ML model ML-M is a U-Net model or a Vision Transformer model. In FIG. 3, the ML model ML-M is embodied as a U-Net model.

Herein, the trained ML model ML-M is trained to ascertain the difference map DA for at least one defined pathological abnormality in the at least one magnetic resonance image MB and/or for an abnormality with regard to at least one defined image quality criterion in the at least one magnetic resonance image MB.

The at least one defined pathological abnormality may for instance comprise a specific pathological abnormality, for example, the presence of a tumor and/or bleeding and/or injuries and/or diseases, etc., on which the ML model ML-M was trained. In an embodiment, the ML model ML-M may be trained for different manifestations of the specific pathological abnormality in the magnetic resonance image data.

Herein, the at least one defined image quality criterion comprises a specific image quality criterion, such as, for example, background noise or signal noise or motion artifacts, etc., in the magnetic resonance image data. In an embodiment, the ML model ML-M may be trained for different manifestations of the specific image quality criterion in the magnetic resonance image data. For example, the ML model ML-M can be trained for magnetic resonance image data from different magnetic resonance sequences with respect to different image quality criteria. For example, for magnetic resonance sequences that are particularly sensitive to background noise in the magnetic resonance images MB, the ML model ML-M can be trained with regard to background noise in the magnetic resonance images MB. In addition, for magnetic resonance sequences in which undesirable movement of the patient frequently occurs, the ML model ML-M can be trained with regard to motion artifacts in the magnetic resonance images MB. Herein, the abnormalities with regard to the at least one defined image quality criterion can comprise a high noise component in the magnetic resonance image data and/or motion artifacts in the image data and/or Gibbs ringing in the magnetic resonance image data. Herein, the defined pathological abnormality and/or the abnormality with regard to a defined image quality criterion is depicted in the difference map DA. Herein, the representation can comprise a color marking in which different deviations from an ideal value can be depicted with different colors in the difference map DA. Alternatively or additionally, the marking can also comprise a border around the defined pathological abnormality and/or the abnormality with regard to a defined image quality criterion in the difference map DA.

A plurality of ML models ML-M can also be trained with respect to different defined pathological abnormalities and/or different defined image quality criteria in order to be able to provide a comprehensive evaluation of the magnetic resonance image MB. Herein, each individual ML model ML-M can be specifically trained to identify one or a few defined pathological abnormalities and/or to identify a defined image quality criterion or a plurality of different defined image quality criteria. In this way, evaluation information BI, for instance a difference map DA or plurality of difference maps DA that covers a plurality of critical image aspects, e.g. a plurality of defined pathological abnormalities and/or a plurality of different defined image quality criteria can be provided for the magnetic resonance image MB and hence alert the medical operator to various potential objections or suspected diagnoses.

Herein, the difference map DA comprises the differences and/or a difference between the reference image RB and the at least one magnetic resonance image MB. The reference image RB may for instance comprise an ideal image comprising no abnormalities or only comprising minor abnormalities, such as artifacts or image noise or signal noise or pathological abnormalities. Herein, the at least one magnetic resonance image MB is embodied as corresponding to the reference image RB. This means that the reference image RB and the at least one magnetic resonance image MB comprise the same image region when mapping the patient and/or also same settings in parameter values of sequence parameters of the measurement sequence to be executed. However, the reference image RB is only required for training the ML model ML-M, e.g. for compiling a training difference map.

In addition to the difference map DA, the evaluation information BI comprises further information, such as, for example, a deviation value AW and an image classification BK (FIG. 3). Herein, the deviation value AW is calculated from the difference map DA, wherein the deviation value AW represents a measure of a pathological abnormality and/or a measure of an image quality in the difference map DA and thus also in the magnetic resonance image MB to be evaluated in relation to the reference image RB. For example, such a deviation value AW can be calculated from a mean value of all pixel values of the difference map DA. The calculation of the deviation value AW may for instance be limited to a relevant region RR of the at least one magnetic resonance image MB. Such a relevant region RR e.g. comprises a mapped organ or part of a mapped organ or a group of mapped organs or a defined mapped body region or even all the mapped anatomical structures, but without background, in the difference map DA. Herein, the relevant region RR can be defined in advance by a user, for example, a physician, for example, as a region RR relevant for the examination. It can also be the case that the relevant region RR is selected depending on a measurement step, e.g. a magnetic resonance sequence. Herein, the selection can also be made at least partially automatically by the trained ML model ML-M.

Herein, the deviation value AW can comprise a value between 0 and 1. Herein, a value of 0 or close to 0 can indicate a small deviation or a minor abnormality in the difference map DA and thus also in the at least one magnetic resonance image MB, while a value of 1 or close to 1 can indicate a major deviation or a major abnormality in the difference map DA and thus also in the at least one magnetic resonance image MB. In addition, the deviation value AW can also comprise a value between −1 and 1.

The image classification BK is ascertained and/or determined from the deviation value AW. In the simplest case, the image classification BK comprises two image classes. For example, the image class “good” can be assigned a deviation value of AW=0 and the image class “bad” can be assigned a deviation value of AW=1. A differentiated image classification BK can have three image classes, wherein the individual image classes can be divided into “good”, “intermediate” and “bad”. Herein, the image class “good” can be assigned a deviation value of AW=0, the image class “intermediate” can be assigned a deviation value of AW=0.5 and the image class “bad” can be assigned a deviation value of AW=1. In addition, further image classes can be available for a particularly differentiated image classification BK. Here, additional image classes can be assigned a deviation value of AW between “good” and “intermediate”, for example, a deviation value AW of 0.3, and a deviation value AW between “intermediate” and “bad”, for example, a deviation value AW of 0.7. In addition to calculating the deviation value AW, the ascertaining and/or determination of an image class for the at least one magnetic resonance image MB is also limited to the relevant region RR.

Subsequently, in a further third method step 102, the evaluation information BI is provided. The evaluation information BI may for instance be provided by means of the evaluation module 50, which has the output interface 52 for this purpose. In an embodiment, the evaluation information BI is provided to output the evaluation information BI to a user, for instance to the medical operator.

In a further subsequent fourth method step 103, the evaluation information BI is output to the user, e.g. to the medical operator, such as, for example, a physician. The evaluation information BI is e.g. output to the medical operator by means of an output unit of the magnetic resonance apparatus 10, preferably a visual output unit, e.g. the display unit 25, such as, for example, a monitor and/or a display.

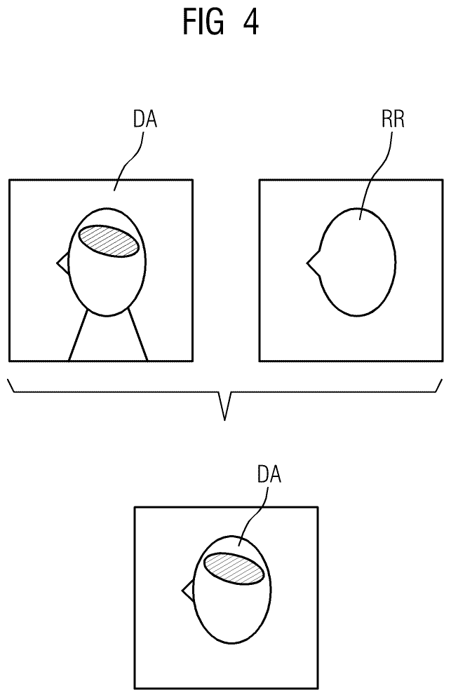

FIGS. 4 to 6 now depict different difference maps DA for different abnormalities in the captured magnetic resonance data. FIG. 4 depicts a first difference map DA provided by the trained ML model ML-M. The difference map DA comprises a map of a head of a patient 16 and of a partial region of the upper body of the patient 16. The head of the patient exhibits an abnormality (gray region). Also shown is a mask representing a relevant region RR, wherein the relevant region RR is limited to the map of the head. The last image in FIG. 4 shows a second difference map DA resulting from a depiction of the first difference map DA with an overlay of the mask with the relevant region RR. Herein, this second difference map DA is limited to the relevant region RR, here the head. Limitation to the relevant region RR enables the abnormality in the difference map DA to be more easily identified by a medical operator.

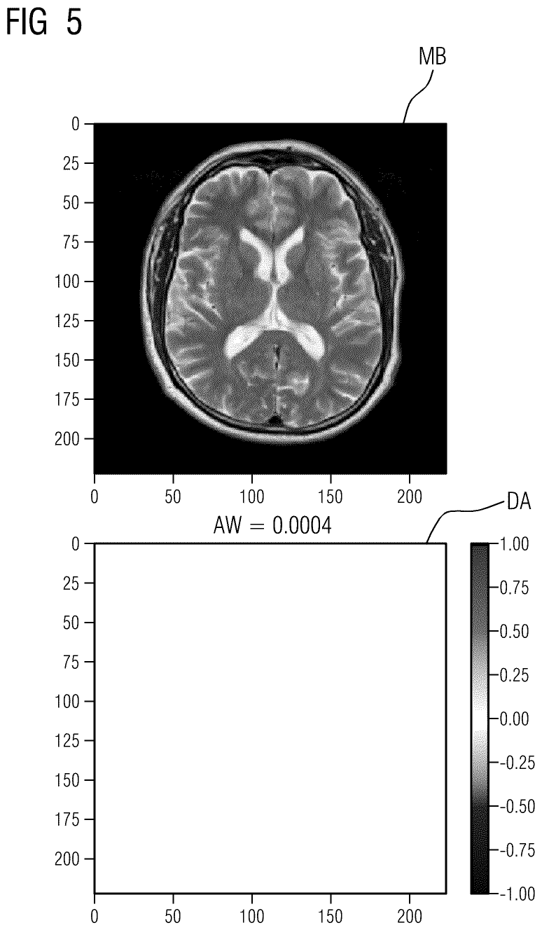

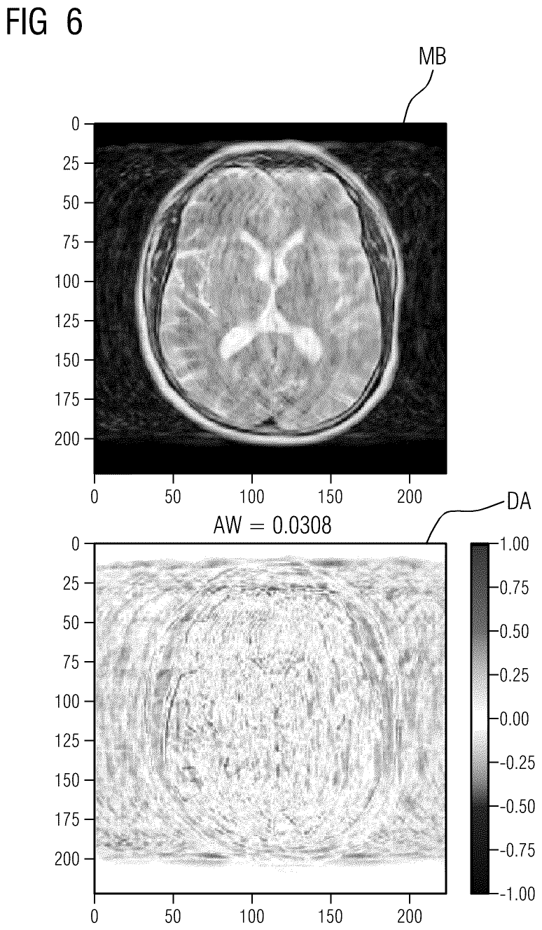

FIGS. 5 and 6 in each case depict magnetic resonance images MB of a head of a patient 16 with associated evaluation information BI. FIG. 5 first depicts an artifact-free magnetic resonance image MB of the head. The evaluation information BI ascertained for the magnetic resonance image MB comprises a difference map DA and a deviation value AW. No abnormalities can be identified in the associated difference map DA ascertained by the trained ML model ML-M. Here, the deviation value AW for the difference map DA calculated therefrom is 0.0004, i.e., almost 0.0 or close to 0.0. Herein, the deviation value AW refers to the relevant region RR, which is limited to the head of the patient 16. Herein, the calculated deviation value AW in FIG. 5 indicates that there are virtually no deviations and/or abnormalities in relation to a magnetic resonance image MB and that a substantially artifact-free magnetic resonance image MB is available for evaluation.

In contrast, FIG. 6 depicts a magnetic resonance image MB of the head with motion artifacts. In addition, FIG. 6 depicts the associated evaluation information BI for the magnetic resonance image MB of the head ascertained by the trained ML model ML-M with the motion artifacts. The evaluation information BI ascertained for the magnetic resonance image MB comprises a difference map DA and a deviation value AW. In this case, the patient 16 moved the head during the data capture, for instance during the capture of the magnetic resonance image MB. The associated difference map DA shows clear abnormalities reflecting the movement of the head during the capture of the magnetic resonance image MB. The greater the deviation from the reference image RB, e.g. the greater and/or stronger a movement of the head of the patient 16, the stronger the color gradient in the difference map DA depicting the deviation. A scale for the color gradient can be seen at the side of the difference map DA. Here, the calculated deviation value AW for the difference map DA is 0.0308.

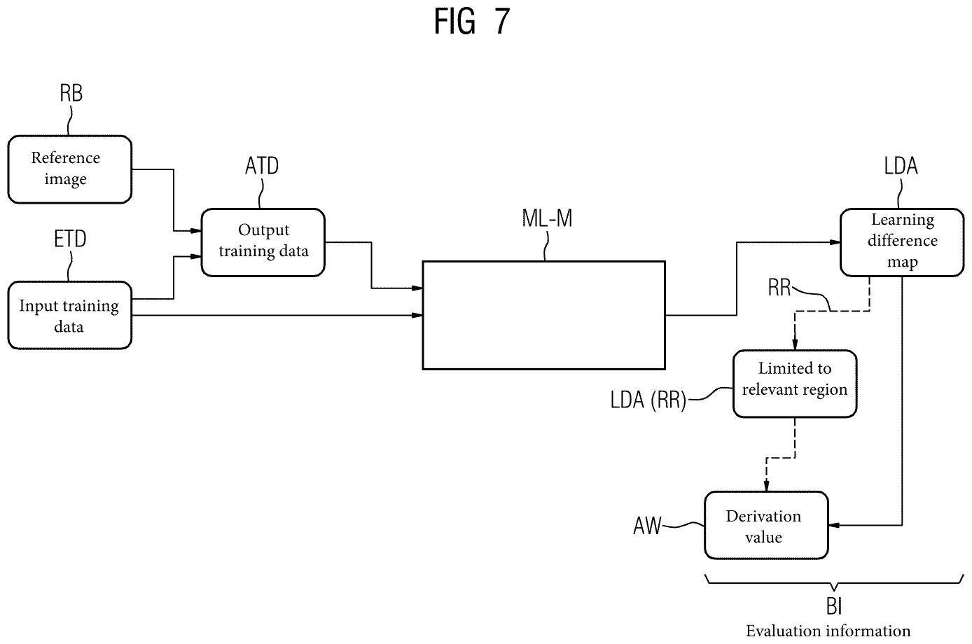

FIG. 7 depicts a method for providing a trained machine-learning model ML-M. Herein, the ML model ML-M is trained to ascertain and provide evaluation information BI comprising a difference map DA for at least one magnetic resonance image MA when executing the method for providing evaluation information BI for at least one magnetic resonance image MB.

Input training data ETD is provided for training the ML model ML-M. The input training data ETD comprises a training magnetic resonance image. In addition, output training data ATD corresponding to the input training data ETD is provided for training the ML model ML-M. The output training data ATD comprises a training difference map. A reference image RB is required to provide the corresponding training difference map, wherein the corresponding training difference map comprises a difference between the training magnetic resonance image and the reference image RB.

The reference image RB basically represents a “good image” and may e.g. comprise an ideal image of magnetic resonance recordings and exhibits no abnormalities or hardly any abnormalities. Herein, this reference image RB can be captured from a healthy patient or a patient who does not make any movements during the capture of magnetic resonance data. Alternatively or additionally, the reference image RB can also be generated by means of a simulation.

If, for example, the ML model ML-M is trained to identify and/or capture motion artifacts in the magnetic resonance images MB, the reference image RB or the plurality of reference images RB should not exhibit any motion artifacts. However, the reference image RB or the plurality of reference images RB can contain other characteristics of a natural data distribution, for example, a wide variety of pathologies and/or other features that affect the image quality, such as, for example, a high noise component in the captured data. If, on the other hand, the ML model ML-M is trained to identify and/or capture a specific and/or defined pathological abnormality in the magnetic resonance images MB, the reference image RB or the plurality of reference images RB should not exhibit this specific pathological abnormality. In contrast, the reference image RB or the plurality of reference images RB can also contain other characteristics of a natural data distribution, such as, for example, motion artifacts or a high noise component or also other pathological abnormalities that do not, however, comprise the defined pathological abnormality.

Furthermore, the training magnetic resonance images are also used to generate and provide the training difference map. These training magnetic resonance images basically represent “bad images” and may e.g. comprise abnormalities with respect to the reference image RB. In an embodiment, abnormalities with regard to a specific image quality criterion are contained in different manifestations, for example from low background noise or signal noise to high background noise or signal noise or from small motion artifacts to strong motion artifacts, in the different training magnetic resonance images. In addition, abnormalities with regard to a specific pathological problem are contained in different manifestations, for example, tumors in different sizes, in the different training magnetic resonance images. Herein, the training magnetic resonance images can be generated on the basis of the reference images RB with the corresponding specific abnormality by means of a simulation algorithm for artifact simulation and/or pathology simulation, so that comparable training magnetic resonance images to the reference images RB are available for the formation of the training difference map DA. Herein, for each reference image RB, e.g. each “good image”, corresponding training magnetic resonance images with abnormalities, e.g. “bad images”, are available that have exactly the same image section. In addition, in this way, the reference images RB, e.g. the “good images”, and the corresponding training magnetic resonance images, for instance the “bad images”, also have the same settings in the sequence parameters. In this way, differences between the training magnetic resonance images and the reference images RB can be assigned to a pathological abnormality and/or an abnormality with regard to an image quality criterion.

The input training data ETD, for instance the training magnetic resonance images, and thus also the training difference maps, exhibit a defined and/or specific image quality problem and/or a defined and/or specific pathological abnormality. However, the input training data ETD, e.g. the training magnetic resonance images, and thus also the training difference maps, can also exhibit more than one defined and/or specific image quality problem and/or more than one defined and/or specific pathological abnormality.

In an embodiment, at least one of the following difference algorithms and/or the following difference metrics is used to ascertain and/or determine the training difference map:
NRMS (normalized root mean square): pixel-by-pixel determination of the difference between “good image” and “bad image”,
MSE (mean squared error),
SSIM (structural similarity index),
PSNR (peak signal-to noise-ratio),
LPIPS (learned perceptual image patch similarity).

Further pixel-based standard image quality metrics and/or difference algorithms for determining the training difference map are conceivable at any time.

Herein, the training of the ML model ML-M is based on the input training data ETD and the output training data ATD. For this purpose, the ML model ML-M is trained to ascertain evaluation information BI for at least one magnetic resonance image MB with regard to a deviation from a reference image RB. Herein, the evaluation information BI ascertained during the learning phase or the training phase comprises training a learning difference map LDA. In an embodiment, the ML model ML-M trained is embodied to make image statements relating to at least one abnormality based on magnetic resonance image data. For example, such an ML model ML-M can be a GAN model or a CycleGAN model. Advantageously, such an ML model ML-M is a U-Net model or a Vision Transformer model. In an embodiment, the ML model ML-M is trained in supervised learning.

The parameters of the trained ML model ML-M can be iteratively adapted to minimize the cost function for a respective batch of training data. For this purpose, gradient optimization algorithms are applied within the ML model ML-M.

Examples of gradient optimization algorithms are:
Adam (Adaptive Moment Estimation):
Adam is an optimization algorithm used for gradient-based optimization of objective functions, e.g. in deep learning. Adam uses exponentially weighted moving averages of both the first and second moments of the gradients for each parameter.

AdaMax (variant of Adam based on infinity norm):
AdaMax is a first order gradient-based optimization method.

SGD (Stochastic Gradient Descent):
SGD is an iterative method for optimizing an objective function with suitable smoothness properties (e.g., differentiable or subdifferentiable). It can be regarded as a stochastic approximation of gradient descent optimization since it replaces the actual gradient (calculated from the entire dataset) by an estimate thereof (calculated from a randomly selected subset of the data).

RMSprop (Root Mean Square Propagation):
RMSprop is an optimization algorithm that scales the learning rate for each parameter based on the magnitude of recent gradients for that parameter.

In addition, for this purpose further state-of-the-art difference algorithms and/or difference metrics can be applied to the learning difference map and the training difference map as the main contribution to the training loss. Examples of such difference algorithms and/or difference metrics include:
NRMS (normalized root mean square): pixel-by-pixel determination of the difference between “good image” and “bad image”,
MSE (mean squared error),
SSIM (structural similarity index),
PSNR (peak signal-to noise-ratio) and
LPIPS (learned perceptual image patch similarity).

Further pixel-based standard image quality metrics and/or standard difference algorithms for determining the evaluation information BI, for instance the learning difference map LDA, are conceivable at any time.

In FIG. 7, during the training and/or the learning phase of the ML model ML-M, first, evaluation information BI comprising the learning difference map LDA with a corresponding deviation value AW is ascertained. This is depicted in FIG. 7 by means of solid arrows. Provided that the evaluation information BI and/or the learning difference map LDA are limited to a relevant region RR by a mask, the evaluation information BI with the learning difference map LDA (RR) is only ascertained for this limitation. In addition, a deviation value AW is also only ascertained for this limitation. The ascertaining of the evaluation information BI with the limitation to a relevant region RR is depicted in FIG. 7 by dashed arrows.

Once the learning phase and/or the training phase of the ML model ML-M is complete, the trained ML model ML-M is provided for use in the method for providing evaluation information BI from at least one magnetic resonance image MB.

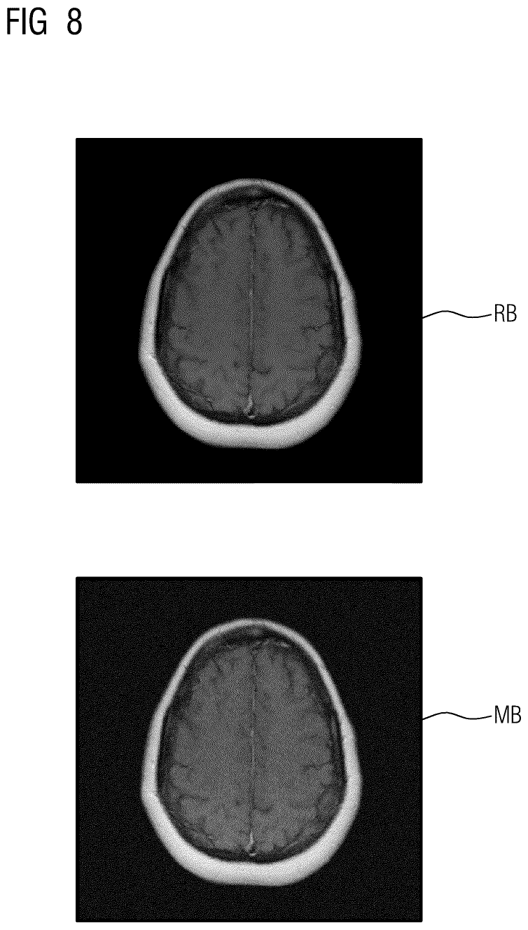

FIG. 8 depicts a reference image RB of a head. Below this, there is a depiction of a magnetic resonance image MB corresponding to the reference image RB with image noise. The corresponding magnetic resonance image MB has been generated from the reference image RB by means of artifact simulation, for instance artifact simulation for generating noise in the image data.

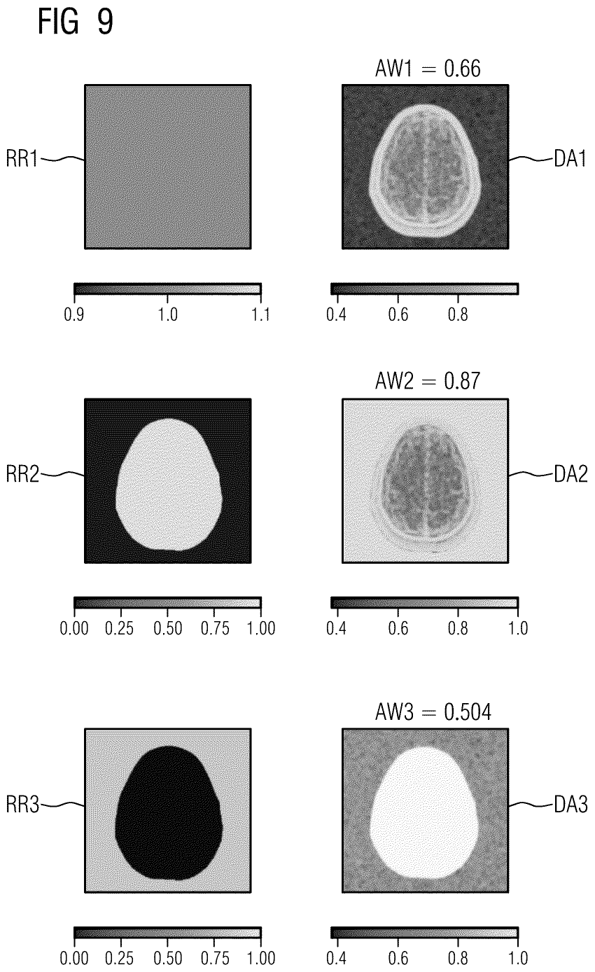

FIG. 9 depicts different items of evaluation information BI (right column) comprising in each case a difference map DA ascertained by means of the trained ML model ML-M and a deviation value AW relative to the reference image RB and the corresponding magnetic resonance image MB from FIG. 8. The difference maps DA were ascertained based on the difference algorithm SSIM. The left column in FIG. 9 in each case shows a relevant region RR to which the difference map DA in the right column refers.

The topmost row depicts a first relevant region RR1 comprising the entire image. Thus, the first difference map DA1 has no restrictions and shows the entire difference map DA. A corresponding first deviation value AW1 is 0.66.

The middle row depicts a second relevant region RR2. This second relevant region RR2 is limited to the head region, so that, in the second difference map DA2, a difference and/or deviation is only ascertained for the head region. Therefore, this second difference map DA2 only depicts a difference and/or deviation for the head region and the background is masked out. Herein, a second deviation value AW2 ascertained for this difference map DA2 is 0.87.

The bottommost row depicts a third relevant region RR3. This third relevant region RR3 is limited to the background and the head region is masked out. This can also be seen in the third difference map DA3, which is limited to a difference and/or deviation for the background. On the other hand, the head region is not depicted in the third difference map DA3. A third deviation value AW3 ascertained for this third difference map DA3 is 0.504.

This demonstrates that limiting the relevant region RR to the head region in which the background is masked out enables a difference and/or deviations to be identified directly in the corresponding difference map DA. In this way, the medical operator, for instance a physician, can also quickly assess the magnetic resonance image MB to be evaluated.

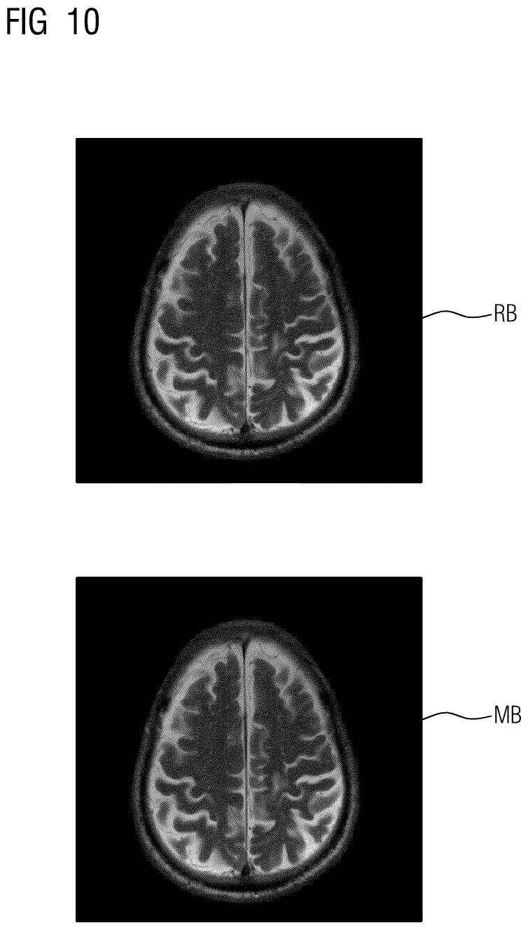

FIG. 10 likewise depicts a reference image RB of a head. Below this, there is a depiction of a magnetic resonance image MB corresponding to the reference image RB with a Gibbs ringing artifact. The corresponding magnetic resonance image MB has been generated from the reference image RB by means of artifact simulation, for instance artifact simulation for generating Gibbs ringing in the image data.

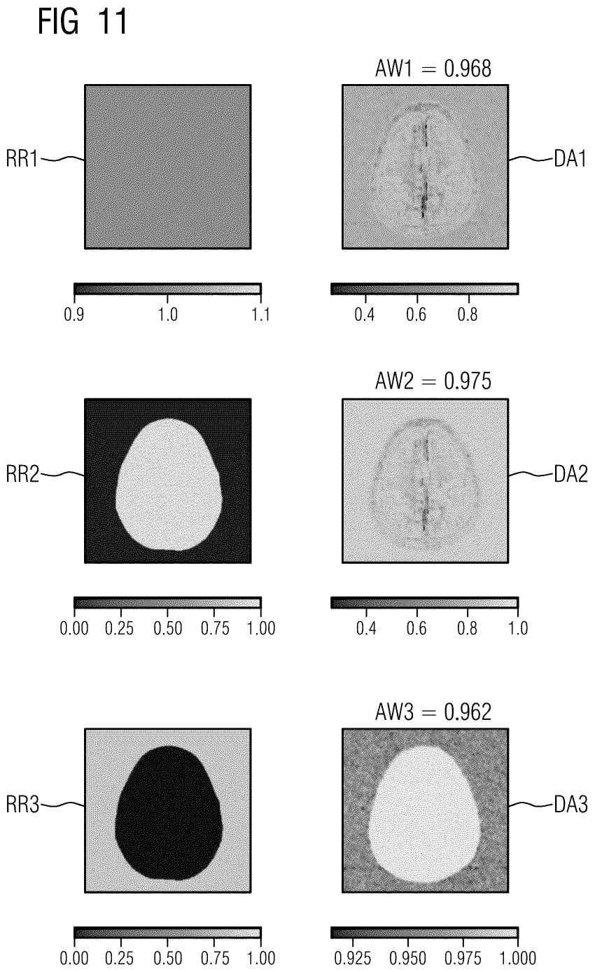

FIG. 11 depicts different items of evaluation information BI (right column) comprising in each case a difference map DA ascertained by means of the trained ML model ML-M and a deviation value AW relative to the reference image RB and the corresponding magnetic resonance image MB from FIG. 10. The difference maps DA were ascertained based on the difference algorithm SSIM. The left column in FIG. 11 shows in each case a relevant region RR to which the difference map DA in the right column refers.

The topmost row depicts a first relevant region RR1 comprising the entire image. Thus, the first difference map DA1 has no restrictions and shows the entire difference map DA. A corresponding deviation value AW is 0.968.

The middle row depicts a second relevant region RR2. The second relevant region RR2 is limited to the head region, so that, in the second difference map DA2, a difference and/or deviation is only ascertained for the head region. Therefore, this second difference map DA2 only depicts a difference and/or deviation for the head region and the background is masked out. Herein, a second deviation value AW2 ascertained for this second difference map DA2 is 0.975.

The bottommost row depicts a third relevant region RR3. This third relevant region RR3 is limited to the background and the head region is masked out. This can also be seen in the corresponding third difference map DA3, which is limited to a difference and/or deviation for the background. On the other hand, the head region is not depicted in the third difference map DA3. Herein, a third deviation value AW3 ascertained for this third difference map DA is 0.962.

This demonstrates that limiting the relevant region RR to the head region in which the background is masked out enables an abnormality of Gibbs ringing artifacts in the magnetic resonance images MB to be clearly and directly identified.

FIG. 12 depicts the evaluation module 50 in more detail. The evaluation module 50 comprises an input interface 51, an output interface 52, and a computing unit 53 (Also referred to herein as processing circuitry or one or more processors). The input interface 51 is embodied to receive magnetic resonance images MB. For this purpose, the input interface 51 communicates with a data interface of a provision module 27 of the magnetic resonance apparatus 10. In addition, the input interface 51 is also embodied to receive the input training data ETD with the training magnetic resonance images and the output training data ATD with the training difference maps during training of the ML model ML-M.

The output interface 52 of the evaluation module 50 is embodied to provide the evaluation information BI ascertained by the evaluation module. The output interface 52 communicates with a data interface of the magnetic resonance apparatus 10, for example a data interface of an output unit, to output the evaluation information BI ascertained by the evaluation module 50 to the medical operator, e.g. display it to the medical operator.

The computing unit 53 of the evaluation module 50 comprises a processor and the ML model ML-M. The processor is embodied to apply the trained ML model ML-M to the magnetic resonance images MB.

Although the disclosure has been illustrated and described in detail by the preferred exemplary embodiment, the disclosure not restricted by the disclosed examples and other variations can be derived herefrom by the person skilled in the art without departing from the scope of protection of the disclosure.

In this text, independent of the grammatical term usage, individuals with male, female or other gender identities are included within the term.

The various components described herein may be referred to as “modules” or “units.” As noted above, such components may be implemented via any suitable combination of hardware and/or software components as applicable and/or known to achieve the intended respective functionality. This may include mechanical and/or electrical components, processors, processing circuitry, or other suitable hardware components configured to execute instructions or computer programs that are stored on a suitable computer readable medium. Regardless of the particular implementation, such modules and units, as applicable and relevant, may alternatively be referred to herein as “circuitry,” “processors,” or “processing circuitry.”

What is claimed is:

1. A computer-implemented method for providing evaluation information from a magnetic resonance image of a patient, comprising:
generating the magnetic resonance image of the patient,
applying a trained machine-learning model to the magnetic resonance image,
generating, via the trained machine-learning model, evaluation information comprising a difference map depicting pixel-by-pixel differences between the magnetic resonance image and a reference image,
wherein the difference map visually marks a pathological abnormality and/or visually marks an abnormality with regard to image quality in the magnetic resonance image; and
outputting the evaluation information including the difference map to a display of a magnetic resonance apparatus during a magnetic resonance examination,
wherein the difference map enables a verification of an evaluation result of the trained machine-learning model.

2. The method as claimed in claim 1, wherein the trained machine-learning model is trained to generate the difference map for:
at least one defined pathological abnormality in the magnetic resonance image; and/or
an abnormality with regard to a defined image quality criterion in the magnetic resonance image.

3. The method as claimed in claim 1, wherein the trained machine-learning model is trained to generate the difference map for an abnormality with regard to a defined image quality criterion comprising signal noise, a motion artifact, or Gibbs ringing in the magnetic resonance image.

4. The method as claimed in claim 1, further comprising:
calculating a deviation value from the difference map,
wherein the deviation value represents a measure of a pathological abnormality and/or of image quality, and
wherein the evaluation information further comprises the deviation value.

5. The method as claimed in claim 4, further comprising:
performing an image classification of the magnetic resonance image comprising at least two image classes based upon the deviation value.

6. The method as claimed in claim 5, wherein (i) the depiction of the difference map, (ii) the calculating of the deviation value, or (iii) the image classification, is limited to a relevant region of the difference map.

7. The method as claimed in claim 1, further comprising:
receiving input training data;
receiving output training data; and
training the machine-learning model based on the input training data and the output training data,
wherein the trained machine-learning model is trained to ascertain the evaluation information from the magnetic resonance image with respect to a deviation from the reference image; and
deploying the trained machine-learning model in the magnetic resonance apparatus.

8. The method as claimed in claim 7, wherein the input training data comprises a training magnetic resonance image, and
wherein the output training data comprises a training difference map.

9. The method as claimed in claim 8, wherein the reference image is generated to provide the training difference map, and
wherein the training difference map is generated from a difference between the training magnetic resonance image and the reference image via a difference algorithm.

10. The method as claimed in claim 8, further comprising:
executing a gradient optimization algorithm to generate a learning difference map.

11. The method as claimed in claim 8, wherein the training magnetic resonance image comprises an abnormality relative to the reference image, and
wherein the training magnetic resonance image is generated from the reference image via artifact simulation and/or pathology simulation.

12. The method as claimed in claim 8, wherein the reference image comprises the same image section as the training magnetic resonance image.

13. The method as claimed in claim 8, wherein the reference image is based on the same sequence parameters as the training magnetic resonance image.

14. The method as claimed in claim 7, wherein the input training data exhibits a defined image quality problem or a defined pathological abnormality.

15. A computing system of a magnetic resonance apparatus, comprising:
an input interface configured to receive a magnetic resonance image;
a memory configured to store a trained machine-learning model;
processing circuitry configured to:
apply the trained machine-learning model to the magnetic resonance image;
generate, via the trained machine-learning model, evaluation information comprising a difference map depicting pixel-by-pixel differences between the magnetic resonance image and a reference image,
wherein the difference map visually marks a pathological abnormality and/or visually marks an abnormality with regard to image quality in the magnetic resonance image; and
an output interface configured to provide the evaluation information including the difference map to a display of the magnetic resonance apparatus during a magnetic resonance examination,
wherein the difference map enables a verification of an evaluation result of the trained machine-learning model.

16. A non-transitory computer-readable medium configured to store instructions thereon that, when executed by processing circuitry of a magnetic resonance apparatus, cause the magnetic resonance apparatus to:
generate a magnetic resonance image of a patient,
apply a trained machine-learning model to the magnetic resonance image,
generate, via the trained machine-learning model, evaluation information comprising a difference map depicting pixel-by-pixel differences between the magnetic resonance image and a reference image,
wherein the difference map visually marks a pathological abnormality and/or visually marks an abnormality with regard to image quality in the magnetic resonance image; and
output the evaluation information including the difference map to a display of the magnetic resonance apparatus during a magnetic resonance examination,
wherein the difference map enables a verification of an evaluation result of the trained machine-learning model.
